(19) United States
(12) Patent Application Publication
(10) Pub. No.: US 2007/0274435 A1
(43) Pub. Date: Nov. 29, 2007

(54) Phase contrast cone-beam CT imaging

(76) Inventors: Ruola Ning, Fairport, NY (US); Weixing Cai, Rochester, NY (US)

Correspondence Address: BLANK ROME LLP, 600 NEW HAMPSHIRE AVENUE, N.W., WASHINGTON, DC, 20037, US

(21) Appl. No.: 11/711,131

(22) Filed: Feb. 27, 2007

Related U.S. Application Data
(60) Provisional application No. 60/776,684, filed on Feb. 27, 2006.

(51) Int. Cl. A61B 6/03; G01N 23/04

(52) U.S. Cl. 378/4; 378/62

(57) ABSTRACT

A cone beam CT imaging system incorporates the phase contrast in-line method, in which the phase coefficient rather than only the attenuation coefficient is used to reconstruct the image. Starting from the interference formula of in-line holography, the terms in the interference formula can be approximately expressed as a line integral that is the requirement for all CBCT algorithms. So, the CBCT reconstruction algorithms, such as the FDK algorithm, can be applied for the in-line holographic projections.

### REFERENCE TO RELATED APPLICATION

The present application claims the benefit of U.S. Provisional Patent Application No. 60/776,684, filed Feb. 27, 2006, whose disclosure is hereby incorporated by reference in its entirety into the present disclosure.

### STATEMENT OF GOVERNMENT INTEREST

The work leading to the present invention was supported in part by NIH Grants 8 R01 EB 002775, R01 9 HL078181, and 4 R33 CA94300. The government has certain rights in the invention.

### FIELD OF THE INVENTION

The present invention is directed to phase-contrast imaging and more particularly to phase-contrast imaging using techniques from in-line holography.

### DESCRIPTION OF RELATED ART

During the last decades, phase-contrast methods have been quickly developed in the x-ray imaging field. Conventionally, x-rays image an object by obtaining a map of only the attenuation coefficient of the object, whereas phase-contrast imaging uses both the phase coefficient and the attenuation coefficient to image an object. Consequently, in the projection image, phase-contrast imaging may resolve some structures that have similar attenuation coefficients but different phase coefficients as their surroundings. In most cases, phase contrast imaging is also an edge-enhancement imaging technique due to its coherence and interference nature. Thus, the boundaries inside small structures could easily be determined. Phase-contrast is a promising technique especially in the case of weak attenuation where the current attenuation-based x-ray CT image might not show sufficient resolution or contrast. Thus, this method could act as an alternative option and/or provide additional information where conventional x-ray imaging fails.

Usually, phase-contrast methods can be classified into three categories. First, the x-ray interferometry method measures the projected phase directly through an interferometer. Second, diffraction-enhanced imaging (DEI) measures the phase gradient along the axial direction. Both of these two methods need not only a synchrotron as a coherent monochromatic x-ray source, but also need relatively complicated optical setups. Third, the in-line holography, essentially measures the Laplacian of the projected phase coefficients. In this case, a micro-focus x-ray tube with a polychromatic x-ray spectrum can be used. The optical setup for in-line holography could be arranged just like a conventional x-ray cone beam CT (CBCT) or a micro-CT. These advantages make it a promising method for practical applications.

Some studies have been conducted for reconstruction schemes using phase-contrast projections. In the DEI and in the in-line holography cases, since the projected phase cannot be measured directly, there are two types of reconstruction. The first type is to retrieve the projected phase coefficients first, and then reconstruct the local phase coefficient for each point in the object area. The second type is to directly reconstruct other related quantities such as the gradient or the Laplacian of the local phase coefficient, instead of retrieving the original phase coefficient.

### SUMMARY OF THE INVENTION

There is thus a need in the art to incorporate the in-line method into current CBCT or micro-CT systems. It is therefore an object of the invention to provide such systems.

To achieve the above and other objects, the present invention is directed to a cone-beam method and system which use the phase coefficient rather than the attenuation coefficient alone to image objects. The present invention may resolve some structures that have similar attenuation coefficients but different phase coefficients relative to their surroundings. Phase contrast imaging is also an edge-enhanced imaging technique. Thus, the boundary of inside small structures could be easily determined.

The present invention incorporates the phase contrast in-line method into current cone beam CT (CBCT) systems. Starting from the interference formula of in-line holography, some mathematical assumptions can be made, and thus, the terms in the interference formula can be approximately expressed as a line integral that is the requirement for all CBCT algorithms. So, the CBCT reconstruction algorithms, such as the FDK algorithm can be applied for the in-line holographic projections, with some mathematical imperfection.

A point x-ray source and a high-resolution detector were assumed for computer simulation. The reconstructions for cone-beam CT imaging were studied. The results showed that all the lesions in the numerical phantom could be observed with an enhanced edges. However, due to the edge-enhancement nature of the in-line holographic projection, the reconstructed images had obvious streak artifacts and numerical errors. The image quality could be improved by using a Hamming window during the filtering process. In the presence of noise, the reconstructions from the in-line holographic projections showed clearer edges than the normal CT reconstructions did. Finally, it was qualitatively illustrated that a small cone angle and weak attenuation were preferred.

Related systems and methods are disclosed in the following U.S. patents: U.S. Pat. No. 6,987,831, “Apparatus and method for cone beam volume computed tomography breast imaging”; U.S. Pat. No. 6,618,466, “Apparatus and method for x-ray scatter reduction and correction for fan beam CT and cone beam volume CT”; U.S. Pat. No. 6,504,892, “System and method for cone beam volume computed tomography using circle-plus-multiple-arc orbit”; U.S. Pat. No. 6,480,565 “Apparatus and method for cone beam volume computed tomography breast imaging”; U.S. Pat. No. 6,477,221, “System and method for fast parallel cone-beam reconstruction using one or more microprocessors”; U.S. Pat. No. 6,298,110, “Cone beam volume CT angiography imaging system and method”; U.S. Pat. No. 6,075,836, “Method of and system for intravenous volume tomographic digital angiography imaging”; and U.S. Pat. No. 5,999,587, “Method of and system for cone-beam tomography reconstruction,” whose disclosures are all incorporated by reference in their entireties into the present disclosure. The techniques disclosed in those patents can be used in conjunction with the techniques disclosed herein.

### BRIEF DESCRIPTION OF THE DRAWINGS

A preferred embodiment of the present invention will be set forth in detail with reference to the drawings, in which:

FIG. 1 is a schematic diagram showing a general scheme for phase-contrast in-line holographic imaging;

FIG. 2 shows that in a 2-D parallel case, the projecting direction is perpendicular to the derivative direction;

FIGS. 3A and 3B show reconstruction slices;

FIGS. 3C-3F show profile plots along the dashed lines in FIGS. 3A and 3B;

FIGS. 4A-4D show reconstruction with Poisson noise imposed to the projections;

FIGS. 5A-5D show the influence of the cone angle on edge enhancement; and

FIGS. 6A-6D show the influence of attenuation on edge enhancement.

### DETAILED DESCRIPTION OF THE PREFERRED EMBODIMENT

A preferred embodiment of the present invention will be set forth in detail with reference to the drawings, in which like reference numerals refer to like elements or steps throughout.

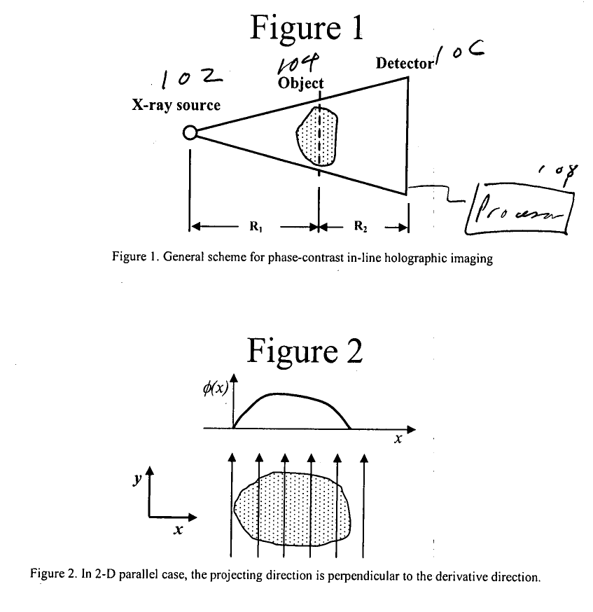

The geometry of the in-line holography is as simple as that of the current mammography or cone beam CT scheme as shown in FIG. 1. A micro-focus x-ray source 102 is placed at a distance R1 from the object 104, and the object is at a distance R₂ from the detector 106. The cone angle should cover the whole region of interest. A processor 108 receives the detected data from the detector 106 and performs the calculations to be described below to produce the image.

In x-ray technology, the refractive index n of a material is usually defined as:
n=1−δ+iβ (1)

where δ is responsible for phase changes and β is related to attenuation. (Physically, δ is proportional to the electron density inside the material, and it is usually 10³ to 10⁴ times larger than β.) Therefore, when a spatially coherent monochromatic x-ray beam travels through a material, its amplitude and phase will be changed. These changes are characterized by the transmission function defined as:
T(x,y)=A(x,y)e^{iφ(x,y)}. (2)

For an object of a finite thickness, the normalized amplitude is given by

$$A ( x , y ) = exp ( - \frac{μ ( x , y )}{2} ) \qquad ( 3 )$$

where

$$μ ( x , y ) = \frac{4 π}{λ} \int β ( x , y , z ) ⅆ z , \qquad ( 4 )$$

and the phase is given by

$$ϕ ( x , y ) = - \frac{2 π}{λ} \int δ ( x , y , z ) ⅆ z . \qquad ( 5 )$$

The integrands of these two line integrals, equations (4) and (5), are integrated over the entire traveling length through the object. It should be pointed out that refraction and diffraction effects might be noticeable when x-ray beams pass through a non-uniform object. Thus, the above line integrals might not be strictly appropriate. Fortunately, it has been shown that if
\sqrt{2λT}<κ, (6)

where T is the maximum thickness of the object and κ is the finest structure size in the object to be imaged, then the object could be regarded as ‘thin’ and the x-ray beams could be considered as traveling along a straight line.

In Wu and Liu's paper (Xizeng Wu, Hong Liu, “Clinical implementation of x-ray phase-contrast imaging: Theoretical foundations and design considerations,” Med. Phys. 30 (8), 2169-2179 (2003)), equations for the in-line holographic projection using both of the attenuation and the phase coefficients were presented. The key result is derived in the case of an ideal point source, showing that with the approximation

$$u ⪡ \sqrt{\frac{M}{λ {R}_{2}}} , \qquad ( 7 )$$

the detected intensity image is expressed as

$$I ( x , y ) = \frac{{I}_{10}}{{M}^{2}} \{ {A}_{0}^{2} ( \frac{x}{M} , \frac{y}{M} ) - \frac{λ {R}_{2}}{2 π M} {∇}^{2} [ {A}_{0}^{2} ( \frac{x}{M} , \frac{y}{M} ) ϕ ( \frac{x}{M} , \frac{y}{M} ) ] \} , \qquad ( 8 )$$

where the definition of I₁₀ is the raw beam intensity, M is the magnification factor, λ is the x-ray wavelength, u is the spatial frequency, A₀² is the amplitude due to attenuation and φ is the projected phase coefficient.

In CBCT or micro-CT imaging, the typical values are M=2, λ=3×10⁻¹¹ m (for 40 keV), R₂=0.5 m, and u is less than 2×10⁴ m⁻¹ (for detector pixel size of 50 μm). Thus, the approximation inequality (7) is satisfied. We can clearly see in equation (8) that the first term in the bracket is related to the attenuation effect, which is being detected by the normal x-ray imaging, while the second term is related to the phase-contrast effect. It should be noticed that in the Laplacian, the projected phase φ is multiplied by the amplitude A₀². Thus, the attenuation coefficient will influence the effect due to the phase part. It has also been proved by experimental data that in weakly attenuating materials, the phase-contrast effect is clearly visible while in strongly attenuating materials, the phase-contrast effect is almost undetectable.

Bearing in mind the in-line holographic projection formulas, conventional CBCT reconstruction algorithms can be applied with these projections after some mathematical manipulation. It is well known that such algorithms as FDK or Radon transform are based on the line integral of local attenuation coefficient. Thus, if a certain type of line integral could be found according to the expression of the exposure intensity in the in-line phase-contrast projection, the FDK algorithms could also be applied. Yet, equation (8) is not a line integral.

Let Equation (8) be rewritten as

$$I ( x , y ) = \frac{{I}_{10}}{{M}^{2}} {A}_{0}^{2} [ 1 - \frac{λ {R}_{2}}{2 π M} \frac{{∇}^{2} ( {A}_{0}^{2} ϕ )}{{A}_{0}^{2}} ] . \qquad ( 9 )$$

Considering the second term in the square bracket and using equation (3), the following is obtained

$$\frac{{∇}^{2} ( {A}^{2} ϕ )}{{A}^{2}} = {∇}^{2} ϕ + [ - {∇}^{2} μ + ∇ \cdot ∇ μ ] ϕ - 2 [ ∇ μ \cdot ∇ ϕ ] . \qquad ( 10 )$$

Since δ is usually 10³ to 10⁴ times larger than β, φ is 10³ to 10⁴ times greater than μ. Therefore, the terms containing μ are negligible in equation (10). That is to say,

$$\frac{{∇}^{2} ( {A}^{2} ϕ )}{{A}^{2}} \approx {∇}^{2} ϕ . \qquad ( 11 )$$

Equation (8) is then reduced to

$$I ( x , y ) = \frac{{I}_{10}}{{M}^{2}} {A}_{0}^{2} [ 1 - \frac{λ {R}_{2}}{2 π M} {∇}^{2} ϕ ] . \qquad ( 12 )$$

If the logarithm is taken on both sides of equation (12), the attenuation part and the phase part could be separated as:

$$ln ( \frac{I}{{I}_{10}} ) + ln {M}^{2} = ln {A}_{0}^{2} + ln [ 1 - \frac{λ {R}_{2}}{2 π M} {∇}^{2} ϕ ] . \qquad ( 13 )$$

In the square bracket, φ is usually on the order 10¹. Thus, for a detector pixel size of 50 μm, the Laplacian is usually no larger than the order 10⁹ m⁻². Given that λR₂ is on the order 10⁻¹¹ m², the second term

$$\frac{λ {R}_{2}}{2 π M} {∇}^{2} ϕ << 1.$$

Equation (13) becomes

$$ln ( \frac{I}{{I}_{10}} ) + ln {M}^{2} = - μ ( x , y ) - \frac{λ {R}_{2}}{2 π M} {∇}^{2} ϕ ( x , y ) . \qquad ( 14 )$$

The first term on the right, μ(x, y), is a line integral while the second term is not a line integral yet. For simplicity, now consider the case of 2-D parallel beam reconstruction for a pure phase phantom (μ=0). Now the projection is only one-dimensional and the 2-D Laplacian is reduced to a 1-D second derivative operator. As shown in FIG. 2, the projecting direction (along the y-axis) is perpendicular to the derivative direction (along the x-axis). Therefore, it is possible to move the second derivative operator into the integrand. In this way, ∇²φ(x, y) becomes the line integral along the y-axis of the second derivative of δ(x, y) at each point inside the 2-D phantom as expressed in equation (15);

$$\frac{{\partial }^{2} ϕ ( x )}{\partial {x}^{2}} = \frac{{\partial }^{2}}{\partial {x}^{2}} ( - \frac{2 π}{λ} \int δ ( x , y ) ⅆ y ) = - \frac{2 π}{λ} \int \frac{{\partial }^{2} δ ( x , y )}{\partial {x}^{2}} ⅆ y . \qquad ( 15 )$$

Thus, the back-projection algorithms can be applied to the parallel-beam geometry. However, it should be noted that when the phantom is illuminated at different angles, the second derivative of each projection is taken at different directions. That is to say, the quantity to be reconstructed at each point varies when the projections are taken at different angles. But for the current back-projection algorithms, it is known that these values should be fixed during the entire process when the whole set of projections is acquired. Intuitively, it could be considered that the reconstructed quantity is the average of the second derivative of δ(x, y) over all directions, rather than the Laplacian itself. In this way, the back-projection algorithm should still work.

In fan-beam or cone-beam geometries, equation (15) is no longer valid because the second derivative direction is usually not perpendicular to the propagating direction of each x-ray beam along which the phantom is projected. In spite of that, if the fan or cone angle is reduced, all the x-ray beams could be considered approximately perpendicular to the detector plane. Subsequently, the detected intensity could be approximately the projected second derivative. Hence, the back-projection algorithms work although the reconstruction is of inferior quality.

To conclude, the result after taking the logarithm is approximately the line integral composed of two parts: the projected attenuation coefficient μ, and the projected Laplacian of the phase coefficient δ averaged over all angular positions. So the in-line holographic projections could be processed by the current reconstruction procedure.

The requirement for detector pixel size is determined by the resolution of the phase-contrast imaging scheme. There are two main factors that affect the resolution. One is the validity of linear propagation. According to equation (6), for typical values in micro-CT as λ˜3×10⁻¹¹ m (40 keV) and T˜0.02 m in current micro-CT applications, the resolution is no better than 2 μm. The second factor is the approximation used in phase-contrast theory as described in equation (7). For M˜2, λ˜3×10⁻¹¹ m and R₂˜0.5 m, equation (8) yields u<<2.5×10⁵ m⁻¹, i.e., where the resolution is much less than 4 μm. Thus, it is reasonable to assume that the resolution is about one tenth of 2.5×10⁵ m⁻¹, which means a detector pixel size of 40-50 μm.

The x-ray source for in-line holography must be spatially coherent. Temporal coherence is not required. That is to say, a polychromatic source is still appropriate. The higher the spatial coherence is, the better the phase contrast results are. In most papers, the spatial coherence is characterized by a coherence length:

$${L}_{coh} = \frac{2 λ {R}_{1}}{s} . \qquad ( 16 )$$

To obtain a large L_{coh}, a small focal spot size (small s) and a large source to object distance (large R₁) are required. λ should not be too large. Otherwise, the projection approximation, equation (6), is not satisfied. Theoretically, the coherence length must be larger than the finest structure to be imaged. For example, if λ=3×10⁻¹¹ m (40 keV), R₁=0.5 m, L_{coh}=25 μm (20 lp/mm, according to the detector pixel size up to a magnification factor M), then the focal spot size s should be no larger than 1.5 μm. It has been proven by both theory and experiments that although L_{coh} is smaller than the size of the finest detail to be imaged, the phase contrast effect would still occur at an inferior quality¹¹. It means that the minimum micro-CT focal spot size, which is around 10 μm, should be small enough for phase-contrast imaging.

In this simulation, an ideal point x-ray source is assumed and a detector pixel size of 50 μm is used.

To incorporate the phase coefficient into the simulation, a modified Shepp-Logan phantom was designed for cone-beam CT geometry. All the geometric parameters are the same as reference 15 up to a factor such that the largest ellipsoid is 18.4 mm in its longest axis. The magnitudes of β and δ are estimated according to their physical properties. According to reference 12, β˜rₑ²ρₑλ and δ˜λ²rₑρₑ, where their ratio is

$$\frac{δ}{β} ∼ \frac{{λ}^{2} {r}_{e} {ρ}_{e}}{{r}_{e}^{2} {ρ}_{e} λ} = \frac{λ}{{r}_{e}} . \qquad ( 17 )$$

The classical electron radius is of the order of 10⁻¹⁵ m. For x-ray photons of energy 40 keV, the wavelength λ is of the order of 10⁻¹¹ m. For water at room temperature, the electron density is about 10³⁰ m⁻³ (approximately 1 mol of water occupies a volume of 18 cm³ and has 6×10²³ molecules, for 10 electrons per molecule.) It can be estimated that β is of the order 10⁻¹¹˜10⁻¹² and δ is about 10⁻⁷˜10⁻⁸.

In CBCT and micro-CT imaging, x-ray photon energies range from 20 keV to 100 keV. Thus, the ratio between δ and β is about 10³ to 10⁴. In this simulation, δ is chosen to be 5000 times larger than β.

The cone beam CT reconstruction is simulated to evaluate the application of FDK algorithm with in-line holographic projections. The simulation parameters are shown in Table 1.

TABLE 1
Simulation parameters of phase-contrast cone beam CT reconstruction

| Photon energy | 20 keV |
|---|---|
| Source-object distance | 0.5 m |
| Source-detector distance | 1.0 m |
| Virtual detector pixel size | (50 μm)³ |
| Number of projections | 360 |
| Reconstruction voxel size | (50 μm)³ |
| Reconstruction dimension | 400 * 400 |
| Fan angle | 3° |

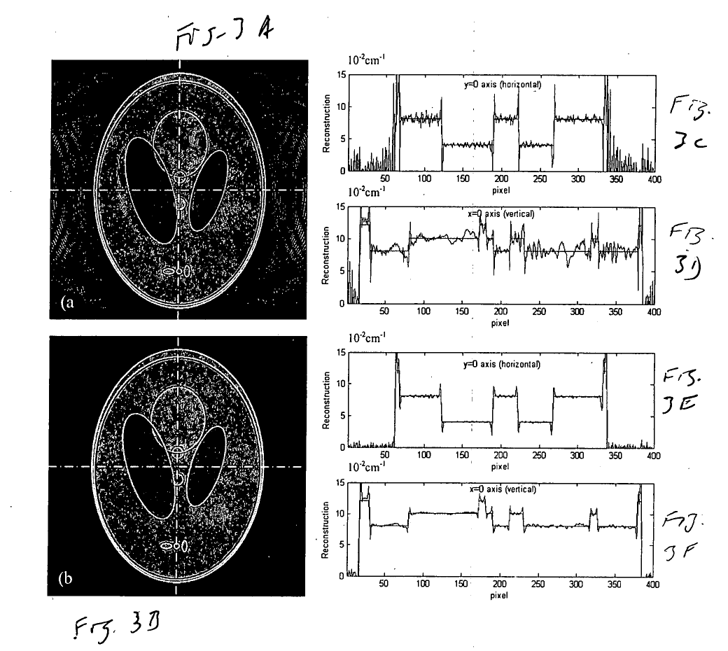

FIGS. 3A-3F illustrate the cone beam reconstruction images and profile plots of the coronal slice at y=−0.25 mm. FIG. 3A shows the reconstruction with a simple ramp filter and the image displays obvious radial-like streak artifacts and numerical distortions. The reason is that the phase-contrast projections themselves have an edge-enhancement nature while the ramp filter tends to magnify the high-frequency component. To suppress the high frequency part and to diminish the artifacts, a Hamming window is added besides the ramp filter during the filtering procedure. As shown in FIG. 3B, in the reconstructed image, the edge enhancement is decreased a little bit, but the artifacts are almost invisible, and the profile looks smoother and better. To demonstrate the edge-enhancement better, the attenuation coefficient is chosen as about one third of that of water. The stronger attenuation case will be discussed later.

FIGS. 3C and 3D show horizontal and vertical profile plots, respectively, along the dashed lines in FIG. 3A. FIGS. 3E and 3F show the horizontal and vertical profile plots, respectively, along the dashed lines in FIG. 3B. The relatively smooth curves are those of the numerical phantom for comparison.

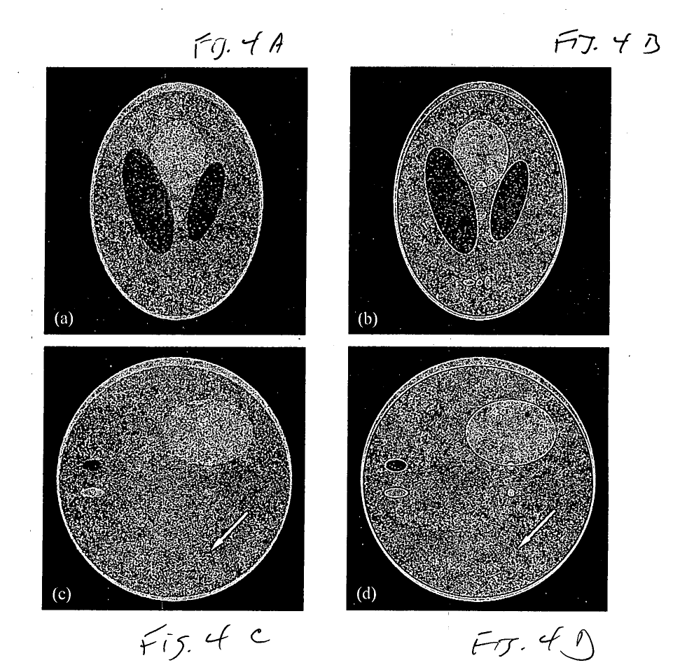

The influence of noise on the reconstruction is studied with the Poisson noise imposed to the projections. The raw x-ray flux is set at 5×10⁶ photons/pixel. Both the coronal slice at y=−0.25 mm and the sagittal slice at x=0.0369 mm are investigated. FIGS. 4A and 4C are normal CBCT reconstruction images. They are so noisy and blurred that the shapes of small structures inside are distorted and the edges are difficult to distinguish from the background. However, in FIGS. 4B and 4D, the reconstruction with in-line holographic projections, all the small structures are clearly observed with enhanced edges. In the sagittal slices, the structure marked by the white arrow cannot be observed in the normal CBCT image but can be seen in the phase-contrast CBCT image.

The degree of edge enhancement due to the phase-contrast effect is determined by several factors. To be compared with the current CT technique, the influences of cone-angle and attenuation to the edge-enhanced effect are qualitatively discussed next.

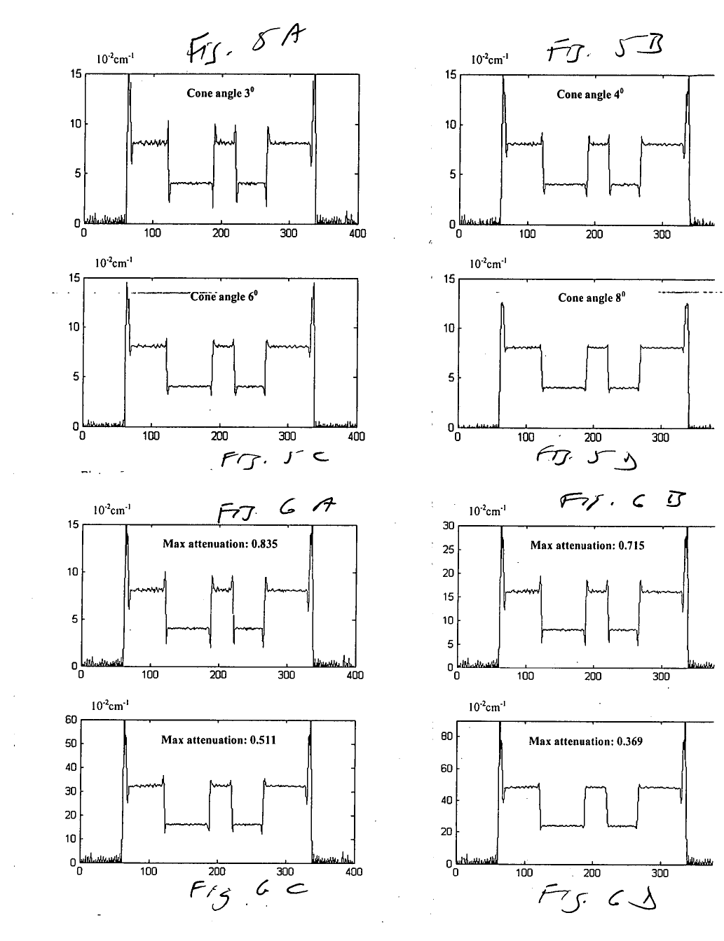

The full cone angle in the above study is set to 3°. As mentioned above, a small cone angle is a better approximation for the line integral of the phase term, while a large cone angle will degrade the edge-enhancement in the reconstruction. To investigate the influence of cone angle to the reconstruction, the object position and the virtual detector pixel size are fixed while the source-to-object distance is adjusted to obtain different cone angles. The slices (y=−0.25 mm) are reconstructed and the horizontal central profiles are plotted for comparison. The reconstructions with four different cone angles are examined, as shown in FIGS. 5A-5D for a cone angle of 3°, 4°, 6° and 8°, respectively, and it is clear that as the angle becomes larger, the edge-enhancement is decreased. When the full cone angle is 6°, the edge-enhancement is still visible. At 8°, little enhancement is achieved.

In the previous simulations, the attenuation coefficient was set rather low in order to clearly demonstrate the edge-enhancement. Here, stronger attenuation cases are considered. In this simulation, all other simulation parameters are the same as before except for the attenuation coefficients and the phase coefficients of the scanned object. They are increased for different attenuation levels. The phase coefficients are modified accordingly to keep the ratio δ/β fixed as before. To illustrate how strong the attenuation is, the minimum detected magnitude (corresponding to the maximum attenuation) in the first projection (at zero degree) is calculated. This value is normalized to the incident x-ray intensity and was used as a measurement of the attenuation strength. In FIGS. 6A-6D, which show the influence of attenuation on edge enhancement, the attenuation measurements in the subplots are 0.835, 0.715, 0.511 and 0.369 respectively. This shows that the edge-enhancement effect decreases with stronger attenuation. The value 0.835 was used with the previous simulations. The value 0.511 is associated with the phantom composed of water at x-ray energy of about 40 keV, and the enhancement is still noticeable. Yet, at 0.369, the enhancement is negligible.

For a small cone angle, the in-line holographic projection could be approximately expressed as a line integral composed of two terms: the projected attenuation coefficient and the projected Laplacian of the phase coefficient. The current CT technology can detect the first term only. The second term can be observed only if the x-ray source is spatially coherent and the detector resolution is high. The FDK algorithm can be applied for the reconstruction of in-line holographic projection data in the cone beam geometry. Due to the edge-enhancement nature of phase-contrast imaging, a Hamming window is necessary in the filtering step to suppress the high-frequency component. Otherwise, the reconstruction will show obvious artifacts and numerical errors. All the structures in the reconstructed images are bounded with enhanced edges when the phase contrast method is applied. The advantage of edge-enhancement is very prominent with the presence of noise. In a normal CT scan, the small structures are blurred and their edges are not clearly identified. Yet, with the phase-contrast effect, all the small structures have clear boundaries. The influence of cone angle size and attenuation is also illustrated. The result shows that the larger the cone angle or the attenuation is, the less the edge-enhancement effect displays, which validates the remarks in the theoretical analysis part. For a phantom of a dimension of around 2 centimeters with a similar attenuation of water, the edge-enhancement is still clearly observed if it is scanned with a full cone angle of less than 5°. Overall in practice, the phase-contrast technique is very promising in micro-CT or small animal imaging.

While a preferred embodiment has been disclosed above, those skilled in the art who have reviewed the present disclosure will readily appreciate that other embodiments can be realized within the scope of the invention. For example, numerical values are illustrative rather than limiting. Also, the invention can be implemented on any suitable scanning device, including any suitable combination of a beam emitter, a flat panel or other two-dimensional detector or other suitable detector, and a gantry for relative movement of the two as needed, as well as a computer for processing the image data to produce images and a suitable output (e.g., display or printer) or storage medium for the images. Software to perform the invention may be supplied in any suitable format over any medium, e.g., a physical medium such as a CD-ROM or a connection over the Internet or an intranet. Therefore, the present invention should be construed as limited only by the appended claims.

We claim:

1. A method for forming an image of an object, the method comprising:
(a) exposing the object to a cone beam of spatially coherent radiation;
(b) receiving the spatially coherent radiation which has passed through the object in a detector to produce detected data;
(c) deriving, from the detected data, an attenuation coefficient and a phase coefficient; and
(d) forming the image from the attenuation coefficient and the phase coefficient.

2. The method of claim 1, wherein step (d) is performed using a cone-beam computed tomography algorithm.

3. The method of claim 2, wherein step (c) comprises filtering the detected data to reduce edge enhancement.

4. The method of claim 3, wherein said filtering comprises suppressing a high-frequency component of the detected data.

5. The method of claim 4, wherein the high-frequency component is suppressed using a Hamming window.

6. The method of claim 2, wherein step (c) comprises deriving a Laplacian of the phase coefficient.

7. The method of claim 2, wherein the spatially coherent radiation is temporally incoherent.

8. The method of claim 2, wherein the spatially coherent radiation has a coherence length which is greater than a size of a finest detail in the object to be imaged.

9. A system for forming an image of an object, the system comprising:
a source of a cone beam of spatially coherent radiation;
a detector for receiving the spatially coherent radiation which has passed through, the object to produce detected data; and
a computer, receiving the detected data, for deriving, from the detected data, an attenuation coefficient and a phase coefficient and forming the image from the attenuation coefficient and the phase coefficient.

10. The system of claim 9, wherein the computer forms the image using a cone-beam computed tomography algorithm.

11. The system of claim 10, wherein the computer filters the detected data to reduce edge enhancement.

12. The system of claim 11, wherein the computer filters the detected data by suppressing a high-frequency component of the detected data.

13. The system of claim 12, wherein the high-frequency component is suppressed using a Hamming window.

14. The system of claim 10, wherein the computer derives a Laplacian of the phase coefficient.

15. The system of claim 10, wherein the spatially coherent radiation is temporally incoherent.
